(19) United States
(12) Patent Application Publication
(10) Pub. No.: US 2018/0003688 A1
(43) Pub. Date: Jan. 4, 2018

(54) FRESHNESS INDICATION

(71) Applicant: KONINKLIJKE PHILIPS N.V., EINDHOVEN (NL)

(72) Inventors: YANLING GAO, EINDHOVEN (NL); DONGHAI YU, EINDHOVEN (NL); RUI KE, EINDHOVEN (NL); NING ZHOU, EINDHOVEN (NL)

(21) Appl. No.: 15/544,571

(22) PCT Filed: Jan. 14, 2016
(86) PCT No.: PCT/EP2016/050590

§ 371 Date: Jul. 19, 2017

(30) Foreign Application Priority Data
Jan. 23, 2015 (CN) PCT/CN2015/071397
Mar. 6, 2015 (EP) 15157939.8

(51) Int. Cl. G01N 33/02; G01N 33/00; G01N 25/56; G01N 21/25; G01N 5/02; G01L 9/08; G01N 21/84; G01B 7/16

(52) U.S. Cl. CPC G01N 33/025; G01N 33/0098; G01N 25/56; G01B 7/16; G01N 5/025; G01L 9/08; G01N 2021/8466; G01N 21/25

(57) ABSTRACT

Embodiments of the present invention relate to freshness indication. In some embodiments, a freshness indicating device is provided. The freshness indicating device comprises a collector and an indicator. The collector is adapted to collect water from an object. The object includes at least one of fruits and vegetables. The indicator is adapted to indicate the freshness of the object based on the amount of the collected water. A corresponding method and a container comprising the freshness indicating device are disclosed as well.

### FIELD OF THE INVENTION

Embodiments of the present invention generally relate to freshness indication, and particularly to a device and method for indicating freshness of fruits and vegetables and to a container capable of indicating freshness of fruits and vegetables stored therein.

### BACKGROUND OF THE INVENTION

Freshness is a quality criterion of great importance to consumers. The detection of the freshness of objects, such as fruits and vegetables, especially during a storage process, fulfils an important need of consumers. By way of example, for consumers, such as ordinary private consumers, vegetable and fruit distributors, raw material managers of restaurants and so on, it is important to detect the freshness of fruits and vegetables to avoid undesired wilting, rotting, and the like.

Traditionally, it is difficult to detect the freshness of fruits and vegetables. By way of example, consumers generally determine whether fruits or vegetables are fresh by observing and smelling. Meanwhile, although some solutions for detecting the freshness have been developed, most of them are invasive, destructive, time-consuming, and specifically workable for lab users instead of ordinary consumers. Additionally, when such developed solutions are employed, certain amounts of chemical wastes would be produced simultaneously, which is also undesirable.

Some earlier patents have shown other attempts for achieving the purpose of freshness detection.

US20120142527A discloses a freshness indicator utilizing a redox sensitive material to indicate the change of the freshness. This redox sensitive material displays different visible properties in its oxidized and reduced forms. Therefore, the degree of the chemical reaction could indicate how long the indicator has been put in the environment, implying the freshness.

U.S. Pat. No. 3,620,677A discloses an indicating device comprising a porous capillary material to induce the progressive absorption of a fluid. In one embodiments, the open end of a sheathed wick is embedded in a frozen food package, the melted water or other liquids formed by thawing causes an upward rise of the liquid in the wick material at a rate proportional to the temperature of the liquid. Accordingly, an accumulation of thawed periods will be permanently recorded on the indicator device reflecting the freshness of the package at the time of use.

WO2011072296A2 discloses a food safety device comprising one or more sensors that are configured to measure at least one condition of the product and/or its environment, for example a humidity sensor. A logic module is configured to execute programmable logic to determine the freshness and/or safety of the product from the at least one measured condition of the product.

However, none of the above prior arts can fully meet the needs of the consumers. In view of the foregoing, there is a need in the art for a solution capable of indicating freshness of fruits and vegetables to consumers in a non-invasive and efficient way.

### SUMMARY OF THE INVENTION

In order to address the above and other potential problems, embodiments of the present invention propose a device and method for indicating freshness of fruits and vegetables, as well as a container capable of indicating freshness of fruits and vegetables stored therein. The invention is defined by the independent claims.

In one aspect, embodiments of the present invention provide a freshness indicating device. The freshness indicating device comprises a collector and an indicator. The collector is adapted to collect water from an object. The object includes at least one of fruits and vegetables. The indicator is adapted to indicate the freshness of the object based on the amount of the collected water.

In another aspect, embodiments of the present invention provide a container. The container comprises a freshness indicating device according to the above aspect.

In still another aspect, embodiments of the present invention provide a method of indicating freshness. The method comprises the steps of: collecting water from an object, the object including at least one of fruits and vegetables; and indicating the freshness of the object based on the amount of the collected water.

Embodiments of the present invention can be implemented to realize one or more of the following advantages. By collecting water from an object belonging to fruits and vegetables, the freshness of the object may be indicated based on the amount of collected water in a non-invasive, low cost and efficient way. As such, it is convenient for consumers, such as ordinary private consumers, vegetable and fruit distributors, and raw material managers of restaurants, to determine whether the object is fresh or not. In this way, the consumers do not have to detect the freshness of fruits and vegetables invasively or inconveniently.

Other features and advantages of embodiments of the present invention will also be understood from the following description of exemplary embodiments when read in conjunction with the accompanying drawings, which illustrate, by way of example, the spirit and principles of the present invention.

### BRIEF DESCRIPTION OF THE DRAWINGS

Details of one or more embodiments of the present invention are set forth in the accompanying drawings and the description below. Other features, aspects, and advantages of the invention will become apparent from the description, the drawings, and the claims, wherein:

FIG. 1 is a schematic diagram illustrating a freshness indicating device in accordance with an exemplary embodiment of the present invention;

FIG. 2A is a schematic diagram illustrating a freshness indicating device in accordance with a further exemplary embodiment of the present invention;

FIG. 2B is a schematic diagram illustrating an embodiment of the freshness indicating device illustrated by FIG. 2A;

FIG. 3 is a schematic diagram illustrating a freshness indicating device in accordance with a still further exemplary embodiment of the present invention;

FIG. 4 is a schematic diagram illustrating a container in accordance with an exemplary embodiment of the present invention;

FIG. 5 is a schematic diagram illustrating a container in accordance with a further exemplary embodiment of the present invention; and

FIG. 6 is a flowchart illustrating a method of indicating freshness in accordance with an exemplary embodiment of the present invention.

Throughout the figures, same or similar reference numbers indicate same or similar elements.

### DETAILED DESCRIPTION OF EMBODIMENTS

The present invention will now be discussed with reference to several example embodiments. It should be understood that these embodiments are discussed only for the purpose of enabling those skilled persons in the art to better understand and thus implement the subject matter described herein, rather than suggesting any limitation on the scope of the subject matter.

The terminology used herein is for the purpose of describing particular embodiments only, and is not intended to be limited to the example embodiments. It will be understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

In general, embodiments of the present invention provide a freshness indicating device and a method of indicating freshness, as well as a container comprising the freshness indicating device. Now some exemplary embodiments of the present invention will be described below with reference to the figures.

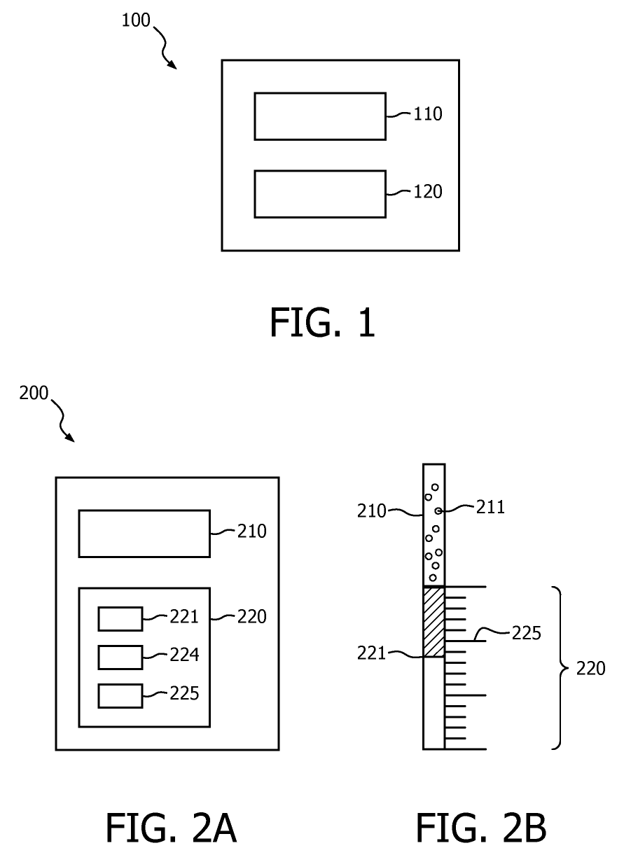

Reference is now made to FIG. 1, where a schematic diagram illustrating a freshness indicating device 100 in accordance with an exemplary embodiment of the present invention is shown.

As shown in FIG. 1, the freshness indicating device 100 comprises a collector 110 and an indicator 120. The collector 110 may be adapted to collect water from an object directly, or indirectly through the environment around the object. The object may include, but is not limited to, one of fruits and vegetables. Fruits and vegetables may have multiple classifications. By way of example, vegetables may include leafy vegetables (such as spinach), root vegetables (such as potato), and so on. Fruits may include fleshy seed-associated structures of a plant that are edible in the raw state, such as apples, oranges, grapes, strawberries, bananas, lemons, and so on. According to embodiments of the present invention, the object may belong to fruits and vegetables. For instance, the object may be spinach, potato(es), apple(s), orange(s) or some combination thereof

It should be noted that, though fruits and vegetables will be described in some embodiments as discussed below, this is merely for the purpose of illustration. Embodiments of the present invention are applicable to e.g. flowers.

The water collected by the collector 110 may contain water vapor, liquid water or a combination thereof. The term “water vapor” refers to the gaseous phase of water. It is one state of water within the hydrosphere. The water vapor may be evaporated from the object. For instance, the water vapor may be produced from respiration of the object, from transpiration of liquid water on the object, or from sublimation of ice on the object. The term “liquid water” refers to water in a liquid state, such as droplets. The liquid water may be, for example, condensed from a portion of water vapor.

The indicator 120 may be adapted to indicate the freshness of the object based on the amount of the collected water. According to embodiments of the present invention, the indicator 120 may be coupled to the collector 110 directly or indirectly. The indicator 120 may have a varied position relative to the collector 110. By way of example, the indicator 120 may be under, above, or parallel to the collector 110.

There is a relationship between freshness and water loss of fruits and vegetables. The relationship may be investigated, for example, by conducting experiments on fruits and vegetables. Taking spinach as a representative, after one week storage in a container, the color of spinach had not changed too much. Therefore, conventionally, it is difficult for consumers to tell the freshness from the visual appearance of the spinach. By investigating water loss, humidity, hardness, and water content of the spinach, the relationship between freshness and water loss may be obtained. By way of example, based on testing results obtained in the experiments, it may be determined that weight loss speed increases with the increase of storage time, and that crispness decrease speed increases with the increase of storage time. Since the crispness decrease speed of spinach has a similar changing trend as water loss, it thus may be determined that freshness and water loss have a linear relationship.

For instance, in some embodiments, the relationship between freshness and water loss of spinach in the refrigerator may be represented by an equation of

y=A·x+B,

where y represents the freshness, x represents the water loss and A and B are constants.

In consideration of the relationship between freshness and water loss, the freshness of the object may be indicated by the indicator 120 based on water collected from the object by the collector 110. As such, it is possible for consumers to obtain the freshness of the object in a non-invasive and efficient way. As a result, undesired invasive means can be avoided, and consumers may conveniently determine freshness of fruits and vegetables at low costs.

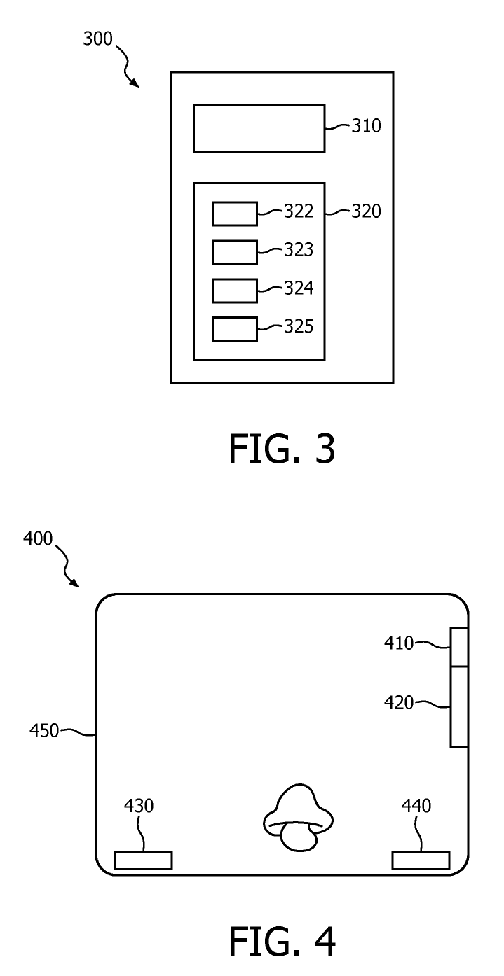

In some embodiments, the indicator may optionally comprise one or more components. For instance, the indicator may comprise a water absorbing element adapted to absorb the collected water, a detecting unit adapted to detect the amount of the collected water, a processing unit adapted to determine the freshness of the object based on the detected amount, a receiving unit adapted to receive information about at least one of classification, weight and temperature of the object, and/or a presenting unit adapted to present the freshness of the object. Details will be described with reference to embodiments of FIGS. 2 and 3.

In some embodiments, the freshness indicating device may optionally comprise a weight sensor adapted to determine the weight of the object, and/or a temperature sensor adapted to sense the temperature of the object. Details will be described with reference to embodiments of FIGS. 2 to 5.

FIG. 2A is a schematic diagram illustrating a freshness indicating device 200 in accordance with a further exemplary embodiment of the present invention. The freshness indicating device 200 as shown in FIG. 2A may be considered as an embodiment of the freshness indicating device 100 described above with reference to FIG. 1. In embodiments described with reference to FIG. 2A, the freshness indicating device 200 comprises a collector 210 and an indicator 220. The indicator 220 further comprises a water absorbing element 221, a receiving unit 224, and a presenting unit 225. However, it is noted that this is only for the purpose of illustrating the principles of the present invention, rather than limiting the scope thereof.

The collector 210 may collect at least one of water vapor and liquid water. For instance, the collector 210 may collect water vapor, liquid water, or a combination of water vapor and liquid water. As discussed above, the water vapor may be evaporated from the object, and the liquid water may be condensed from a portion of the water vapor.

In some embodiments, the collector 210 may comprise a tube, a box or any other suitable element in which a space is reserved for collecting water vapor and liquid water from the object. The tube may be, for example, a capillary tube which is hollow and made of hydrophobic materials, such that the condensed droplets can easily roll off. The collector 210 may be placed in a container accommodating the object, and the space of the collector 210 may be in contact with a space of the container. When water vapor is evaporated from the object, it may spread to the whole space of the container. Accordingly, the space of the collector 210 receives the water vapor from the object. In some cases, the collected water vapor may condense into droplets due to a number of factors, such as temperature decrease, equilibration, and so on. Thus, liquid water condensed from a portion of the water vapor may be also collected by the collector 210.

On the basis of the amount of water collected by the collector 210, the indicator 220 may indicate the freshness of the object. In some embodiments, the indicator 220 may comprise a water absorbing element 221. The water absorbing element 221 may have the property of presenting different states with respect to different amounts of water. As such, when the water absorbing element 221 absorbs water collected by the collector 210, it may present different states, such as different colors, sizes, or shapes, dependent on the amount of the collected water. In an embodiment, the water absorbing element 221 may comprise silica gel materials with or without cobalt, for example, a silica gel column. Upon absorbing the water collected by the collector 210, the water absorbing element 221 may change color accordingly. For instance, as for a water absorbing element 221 comprising silica gel with cobalt, the color may change from purple to blue, and as for a water absorbing element 221 comprising cobalt-free silica gel, the color may change from orange to green.

Optionally, the indicator 220 may comprise a receiving unit 224 adapted to receive information about at least one of classification, weight and temperature of the object, which may be, for example, inputted by consumers. It is to be noted that the receiving unit 224 is optional, rather than essential. For example, in an alternative embodiment, the information may be preset to the freshness indicating device 200. In this regard, the freshness indicating device 200 may be implemented without the receiving unit 224. It is further to be noted that, in addition to classification, weight, and temperature of the object, the information may include other properties of the object that may have influence on the determination of the freshness. The above examples merely serve for the purpose of illustration, rather than limitation.

The freshness of the object may be expressed by, for example, levels, scores, percentages and so on. In some embodiments, the indicator 220 may comprise a presenting unit 225 adapted to present the freshness of the object. The presenting unit 225 may present the freshness visually or audibly. In an embodiment, the presenting unit 225 may have a scale. The scale may mark different levels of freshness, such that the freshness may be presented visually. The levels of freshness may be designed as being associated with different degrees of status change. According to some embodiments of the present invention, the levels of freshness may be determined based on information about the object, such as classification, weight, temperature, which may be preset or which may be received from the receiving unit 224. When the water absorbing element 221 absorbs water and presents different statuses (for example, different colors) with respect to different amounts of water, the freshness of the object may be clearly reflected by the scale and the degree of color change. For instance, if the scale has five levels of freshness, that is, levels 1 to 5, and if half of the water absorbing element 221 has changed color, for instance, from orange to green, it may be determined that the freshness of the object corresponds to the intermediate level, that is, level 3. In this way, the freshness of the object may be indicated to consumers concisely.

Reference is now made to FIG. 2B, which is a schematic diagram illustrating an embodiment of the freshness indicating device 200. As shown in FIG. 2B, the collector 210 collects water 211, such as water vapor and liquid water, from an object. The indicator 220 comprises a water absorbing element 221 and a display unit 225. The water absorbing element 221 absorbs water collected by the collector 210 and changes its colors, shown in a hatched portion. The presenting unit 225 has a scale that marks different levels of freshness. In this way, the degree of the color change may be reflected by the scale. As a result, the freshness of the object may be presented visually.

It should be also understood that the scale may be designed in any other suitable way as long as it is capable of reflecting the amount of water directly or indirectly. For example, the scale may be associated with different statuses, such as size and shape, and the scale may be implemented with a LCD, on which levels of freshness may be varied in terms of different classifications, weights, temperatures of the object. It is further to be noted that the levels of freshness marked on the scale may be designed in any other suitable way. The above examples are shown only for illustrative purposes, without suggesting any limitations on the scope of the subject matter described herein.

Alternatively or additionally, the presenting unit 225 may have a display for visually presenting the freshness. The display may comprise, but is not limited to, a Liquid Crystal Display (LCD), light-emitting diodes (LED), and/or any other suitable display device that has been developed and/or that will be developed in the future.

Alternatively or additionally, the presenting unit 225 may have a speaker for audibly presenting the freshness, for example, by beeps, human voice, music, and the like.

In addition to the forgoing, in some embodiments, the freshness indicating device 200 may optionally comprise a weight sensor adapted to determine the weight of the object.

By means of the weight sensor, the freshness indicating device 200 may obtain the weight of the object before or after storage. In an embodiment, the weight sensor may be coupled to the indicator 220 to provide the weight information of the object.

In some embodiments, the freshness indicating device 200 may optionally comprise a temperature sensor adapted to sense the temperature of the object. The temperature of the object may be the temperature at which the object is stored, or the temperature inside a container storing the object. By means of the temperature sensor, the freshness indicating device 200 may obtain the temperature of the object. In an embodiment, the temperature sensor may be coupled to the indicator 220 to provide information about the temperature of the object.

FIG. 3 is a schematic diagram illustrating a freshness indicating device 300 in accordance with a still further exemplary embodiment of the present invention. The freshness indicating device 300 as shown in FIG. 3 may be considered as an embodiment of the freshness indicating device 100 described above with reference to FIG. 1. In embodiments described with reference to FIG. 3, the freshness indicating device 300 comprises a collector 310 and an indicator 320. The indicator 320 further comprises a detecting unit 322, a processing unit 323, a receiving unit 324, and a presenting unit 325. However, it is noted that this is only for the purpose of illustrating the principles of the present invention, rather than limiting the scope thereof

In the embodiments of FIG. 3, the collector 310 may comprise water absorbing materials, such as hydrogel. The collector 310 may be arranged in a container accommodating the object, for example, located at a wall of the container. When water vapor is evaporated from the object, it may condense into droplets due to a temperature decrease, equilibration, and so on. In this regard, the collector 310 may collect liquid water by absorbing the condensed droplets.

On the basis of the amount of water collected by the collector 310, the indicator 320 may indicate the freshness of the object. In some embodiments, the indicator 320 may comprise a detecting unit 322. The detecting unit 322 may be coupled to the collector 310 and adapted to detect the amount of the collected water. In an embodiment, the detecting unit 322 may comprise a piezoelectric film. The piezoelectric film may produce a voltage in proportion to compressive or tensile mechanical stress or strain, and may be for example made of Polyvinylidene Fluoride (PVDF), which is highly sensitive, flexible, stable and lightweight. In the case that the collector 310 collects liquid water by absorbing condensed droplets, it may exert a pressure on the detecting unit 322. Upon receiving the pressure, the detecting unit 322 may generate an electrical signal indicating the amount of the collected water.

In some embodiments, the indicator 320 may comprise a processing unit 323 coupled to the detecting unit 322. The processing unit 323 may determine the freshness of the object based on the detected amount. In some optional embodiments, the processing unit 323 may obtain information about the object, so that the freshness may be determined more accurately. The information may be, for example, but is not limited to, classification, weight, temperature of the object and/or a combination thereof. As discussed, there is a relationship between freshness and water loss of fruits and vegetables. In practice, the relationship may be affected by several factors, such as classification, weight, temperature of the object. By way of example, for different classifications of fruits and vegetables, such as leafy vegetables and root vegetables, even if they have the same water loss, the freshness may be different.

In another example, for different weights of one classification of fruits and vegetables, such as spinach in an amount of 1 kg and 2 kg, if they have the same water loss, the freshness may be different. In yet another example, for different temperatures of an object, such as, 4° C. and 25° C., the relationship between freshness and water loss may be different. As such, on the basis of the obtained information such as classification, weight, temperature of the object, as well as the amount of the collected water, the processing unit 323 may determine the freshness of the object in a more accurate way.

Optionally, the indicator 320 may comprise a receiving unit 324 adapted to receive information about at least one of classification, weight and temperature of the object, which may be, for example, inputted by consumers. In some embodiments, the receiving unit 324 may be coupled to the processing unit 323, such that the processing unit 323 may obtain the information from the receiving unit 324. It is to be noted that the receiving unit 324 is optional, rather than essential. In alternative embodiments, the information may be preset to the freshness indicating device 300. In such a case, the freshness indicating device 300 may be implemented without the receiving unit 324. It is further to be noted that, in addition to classification, weight, and temperature of the object, the information may include other properties of the object that may have an influence on the determination of the freshness. The above examples merely serve for the purpose of illustration, rather than limitation.

The freshness of the object may be expressed by, for example, levels, scores, percentages and so on. In some embodiments, the indicator 320 may comprise a presenting unit 325 adapted to present the freshness of the object. The presenting unit 325 may present the freshness visually or audibly. By way of example, the presenting unit 325 may have a speaker which may audibly present the freshness to consumers, for example, by beeps, human voice, music, and the like. Alternatively or additionally, the presenting unit 325 may have a display which may visually present the freshness. The display may comprise, but is not limited to, a LCD, LEDs, and/or any other suitable display device that has been developed and/or that will be developed in the future.

In some embodiments of the present invention, the freshness indicating device 300 may optionally comprise a weight sensor adapted to determine the weight of the object. By means of the weight sensor, the freshness indicating device 300 may obtain the weight of the object before or after storage. In an embodiment, the weight sensor may be coupled to the indicator 310, for example, the processing unit 323, to provide the information about weight of the object.

In some embodiments of the present invention, the freshness indicating device 300 may optionally comprise a temperature sensor adapted to sense the temperature of the object. The temperature of the object may be the temperature at which the object is stored, or the temperature inside a container storing the object. By means of the temperature sensor, the freshness indicating device 300 may obtain the temperature of the object. In an embodiment, the temperature sensor may be coupled to the indicator 310, for example, the processing unit 323, to provide information about the temperature of the object.

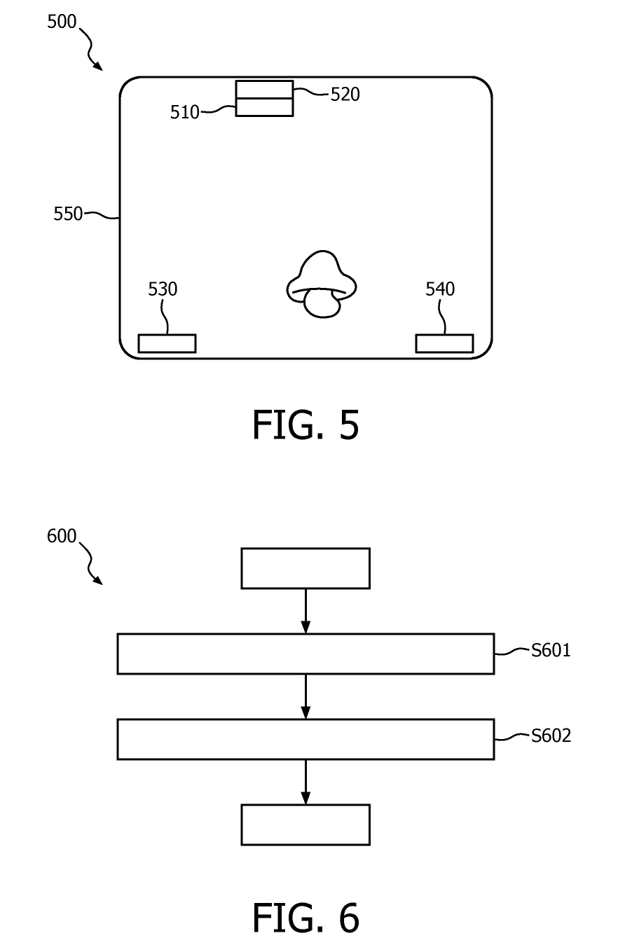

According to embodiments of the present invention, the freshness indicating device according to embodiments of the present invention, such as the freshness indicating device 100, 200 or 300, may be used alone or with a container, which is used for storing fruits and vegetables. Now some exemplary embodiments of the container will be described below with reference to FIGS. 4 and 5.

Reference is now made to FIG. 4, which is a schematic diagram illustrating a container 400 in accordance with an exemplary embodiment of the present invention.

According to embodiments of the present invention, the container 400 may be any type of flexible, semi-rigid, or rigid container. By way of example, the container 400 may be a plastic bag, a plastic casing, a crisper, and so on. Furthermore, the container 400 may be implemented in a refrigerator, a refrigeration house, and other suitable storage environments.

The container 400 may have various shapes, such as cuboid, sphere, ellipsoid and some other regular or irregular shapes. In the case that the container 400 has the shape of a cuboid, it may comprise a wall 450 and a base. It is to be noted that the base is not essential for the container 400. For example, in the case that the container 400 has the shape of a sphere or ellipsoid, it only has a wall 450, without the base. The wall 450 of the container 400 defines an accommodating space. The accommodating space may accommodate fruits or vegetables which need to be stored.

As shown in FIG. 4, the container 400 comprises a freshness indicating device as described in embodiments of the present invention. More specifically, the freshness indicating device comprised by the container 400 comprises a collector 410 and an indicator 420, which may be similar to the collector 210 and the indicator 220, as discussed with reference to FIG. 2A. The collector 410 may be adapted to collect water from an object, for example (a) mushroom(s), stored in the container 400. The collector 410 may comprise a tube, a box or any other suitable element in which a space is reserved for collecting water vapor and liquid water from the object, wherein the water vapor is evaporated from the object, and the liquid water is condensed from a portion of the water vapor.

The indicator 420 may be adapted to indicate the freshness of the object based on the amount of the water collected by the collector 410. The indicator 420 may comprise a water absorbing element, which absorbs water collected by the collector 410 and which presents different colors with respect to different amounts of water. The freshness may be represented by a plurality of levels of a scale. The levels may be designed as being associated with different degrees of color change. In an embodiment where the water absorbing element comprises cobalt-free silica gel, if the scale has five levels of freshness, that is, levels 1 to 5, and if half of the water absorbing element has changed color, for instance, from orange to green, it may be determined that the freshness of the object corresponds to level 3. In this way, the freshness of the object may be indicated to consumers concisely.

In embodiments described with reference to FIG. 4, the freshness indicating device may further comprise a weight sensor 430 and a temperature sensor 440, which are arranged at the bottom of the container 400. It should be also understood that the location of the weight sensor 430 may be changed as long as it is capable of determining the weight of the object. It should be further understood that the location of the temperature sensor 440 may be changed as long as it is capable of sensing the temperature of the object. The above examples are shown only for illustration purposes, without suggesting any limitations on the scope of the subject matter described herein.

By means of the container 400 comprising the freshness indicating device according to embodiments of the present invention, consumers may determine the freshness of fruits and vegetables stored in the container 400 in a non-invasive and efficient way.

Reference is now made to FIG. 5, which is a schematic diagram illustrating a container 500 in accordance with a further exemplary embodiment of the present invention.

Similar to the container 400, the container 500 may be any type of flexible, semi-rigid, or rigid container. By way of example, the container 500 may be a plastic bag, a plastic casing, a crisper, and so on. Furthermore, the container 500 may be implemented in a refrigerator, a refrigeration house, and other suitable storage environments. The container 500 may have various shapes, such as cuboid, sphere, ellipsoid and some other regular or irregular shapes, and may have a wall 550 defining an accommodating space in which fruits or vegetables may be stored.

As shown in FIG. 5, the container 500 comprises a freshness indicating device including a collector 510 and an indicator 520, which may be similar to the collector 310 and indicator 320. The collector 510 may be arranged inside the container 500 and adapted to collect water from an object, for example (a) mushroom(s), stored in the container 500. The collector 510 may comprise water absorbing materials, such as hydrogel, so as to collect liquid water that is condensed from a portion of water vapor evaporated from the object.

The indicator 520 may be arranged on the inner wall of the container 500. The indicator 520 may comprise a detecting unit coupled to the collector 510 and adapted to detect the amount of the collected water. In embodiments described with reference to FIG. 5, the detecting unit may comprise a piezoelectric film which is, for example, made of PVDF. The piezoelectric film may produce a voltage in proportion to compressive or tensile mechanical stress or strain. Since the collector 510 collects liquid water by absorbing condensed droplets, it may exert a pressure on the detecting unit. Upon receiving the pressure, the detecting unit may generate an electrical signal indicating the amount of the collected water.

In embodiments described with reference to FIG. 5, the indicator 520 may further comprise a processing unit which determines the freshness of the object based on the detected amount of water. In some embodiments, the processing unit may obtain information about the object, such as classification, weight, temperature of the object and/or a combination thereof, so that the freshness may be determined more accurately. The information about the object may be predefined by consumers, or may be inputted by consumers in real time. For the latter, the indicator 520 may further comprise a receiving unit adapted to receive information about the object.

When the freshness of the object has been determined, the indicator 520 may present the freshness visually or audibly, for example, by using a presenting unit. The freshness may be expressed by levels, scores, percentages and so on. In some embodiments, the freshness may be audibly presented by a speaker which may produce beeps, human voice, music, and the like. In some other embodiments, the freshness may be visually presented by a display, such as, a LCD, LEDs, and/or any other suitable display device that has been developed and/or that will be developed in the future.

In embodiments described with reference to FIG. 5, the freshness indicating device may further comprise a weight sensor 530 adapted to determine the weight of the object and a temperature sensor 540 adapted to sense the temperature of the object. By means of the container 500 comprising the freshness indicating device according to embodiments of the present invention, consumers may determine the freshness of fruits and vegetables stored in the container 500 in a non-invasive and efficient way.

The freshness indicating devices illustrated in the container 400 and 500 may be considered as embodiments of the freshness indicating devices 200 and 300, respectively. It is to be noted that this is shown only for illustration purpose, without suggesting any limitations on the scope of the subject matter described herein. Any other suitable variant of the freshness indicating device according to embodiments of the present invention may be applicable to the container.

It is further to be noted that the containers 400 and 500 may comprise other units or devices implemented by any suitable technologies, either currently known or to be developed in the future, which will not be detailed here. The above examples are shown only for illustration purpose, without suggesting any limitations on the scope of the subject matter described herein.

Reference is now made to FIG. 6, which is a flowchart illustrating a method 600 of indicating freshness in accordance with exemplary embodiments of the present invention. The method 600 may be performed by means of the freshness indicating device according to embodiments of the present invention, for example those discussed with respect to FIGS. 1 to 5, as well as various modifications, adaptations to the foregoing exemplary embodiments.

The method 600 begins at step S601, where water is collected from an object. Step S601 may be performed by a collector of a freshness indicating device according to embodiments of the present invention. The object may include, but is not limited to, one of fruits and vegetables.

The water collected at step S601 may contain water vapor, liquid water or a combination thereof. The water vapor may be evaporated from the object, and the liquid water may be condensed from a portion of water vapor. In some embodiments, both water vapor and liquid water may be collected into a collector, such as a tube, a box or any other suitable element in which a space is reserved for collecting water vapor and liquid water.

Alternatively, in some embodiments, only liquid water may be collected, for example, by absorbing the liquid water using water absorbing materials, such as hydrogel.

At step S602, the freshness of the object is indicated based on the amount of the collected water. Step S602 may be performed by an indicator of the freshness indicating device.

In some embodiments, the indicator may absorb water collected at step S601 and present different statuses, for example, change color, shape or size, with respect to a different amount of water. Additionally, the indicator may mark different levels of freshness, for example, by using a scale. The levels of freshness may be associated with different colors, shapes and/or sizes. As such, the freshness of the object may be reflected by the scale when a different status is presented.

Alternatively, in some embodiments, the indicator may detect the amount of water collected at step S601, determine the freshness of the object based on the detected amount, and present the freshness. By way of example, at step S601, liquid water condensed from water vapor evaporated from the object may be absorbed by, for example, hydrogel. The hydrogel thus may exert pressure on, for example, piezoelectric polymer, to produce a voltage in proportion to the exerted pressure. As such, at step S602, the amount of the collected water may be detected. Based on the detected amount, the freshness may be determined by considering the relationship between freshness and water loss of fruits and vegetables. In the process of determination of the freshness, some additional information, for example, classification, weight, temperature of the object, may be taken into account. Then, the determined freshness may be presented audibly and/or visually, for example, by using a speaker and/or a display.

While this specification contains many specific implementation details, these should not be construed as limitations on the scope of any disclosure or of what may be claimed, but rather as descriptions of features that may be specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described, as is the case hereinabove, as acting in certain combinations and may even be initially claimed as such, one or more features of a claimed combination can in some cases be excised from the combination, and the claimed combination may be directed to a sub-combination or a variation of a sub-combination.

Various modifications, adaptations to the foregoing exemplary embodiments of this invention may become apparent to those skilled in the relevant arts in view of the foregoing description, when read in conjunction with the accompanying drawings. Any and all modifications will still fall within the scope of the non-limiting and exemplary embodiments of this invention. Furthermore, other embodiments of the invention set forth herein will come to mind of one skilled in the art to which these embodiments of the invention pertain, having the benefit of the teachings presented in the foregoing descriptions and the drawings.

Therefore, it will be appreciated that the embodiments of the invention are not to be limited to the specific embodiments disclosed and that modifications and other embodiments are intended to be included within the scope of the appended claims. Although specific terms are used herein, they are used in a generic and descriptive sense only and not for purposes of limitation.

1. A freshness indicating device, comprising:
a collector adapted to collect water from an object, the object being at least one of fruits, vegetables, flowers and mushrooms; and
an indicator adapted to indicate the freshness of the object based on the amount of the collected water, wherein the indicator includes a water absorbing element adapted to absorb the collected water, a property of the water absorbing element being that ir presents different states with respect to different amounts of water,
wherein the collector is arranged for collecting water resulting from water lost by the object while freshness is decreasing.

2. The device according to claim 1, wherein the collector is adapted to collect at least one of water vapor and liquid water, the water vapor being evaporated from the object, and the liquid water being condensed from a portion of the water vapor.

3. (canceled)

4. The device according to claim 1, wherein the property includes at least one of color, size and shape.

5. The device according to claim 1, wherein the indicator further includes:
a detecting unit adapted to detect the amount of the collected water.

6. The device according to claim 5, wherein the indicator further includes:
a processing unit adapted to determine the freshness of the object based on the detected amount.

7. The device according to claim 6, wherein the processing unit is further adapted to obtain information about at least one of classification, weight and temperature of the object, and determine the freshness of the object based on the information and the amount of the collected water.

8. The device according to claim 1, wherein the indicator further includes:
a receiving unit adapted to receive information about at least one of classification, weight and temperature of the object.

9. The device according to claim 1, wherein the indicator further includes:
a presenting unit adapted to present the freshness of the object.

10. The device according to claim 1, further comprising:
a weight sensor adapted to determine the weight of the object.

11. The device according to claim 1, further comprising:
a temperature sensor adapted to sense the temperature of the object.

12. A container (=comprising
a wall forming an accommodating space for an object, the object including at least one of fruits and vegetables; and
a freshness indicating device according to claim 1.

13. A method of indicating freshness, the method comprising the steps of:
collecting water from an object, the object being at least one of fruits, vegetables, flowers, and mushrooms; and
indicating the freshness of the object based on the amount of the collected water,
wherein the step of indicating the freshness of the object comprises the step of absorbing the collected water and presenting a different states of a property with respect to different amounts of the collected water, and wherein the collected water results from water lost by the object while freshness is decreasing.

14. The method according to claim 13, wherein the step of collecting water comprises a step of collecting at least one of water vapor and liquid water, the water vapor being evaporated from the object, and the liquid water being condensed from a portion of the water vapor.

15. The method according to claim 13, wherein the step of indicating the freshness of the object based on the amount of the collected water further comprises the steps of:
detecting the amount of the collected water;
determining the freshness of the object based on the amount of the collected water; and
presenting the freshness of the object.
